(19) United States
(12) Patent Application Publication
(10) Pub. No.: US 2023/0187087 A1
(43) Pub. Date: Jun. 15, 2023

(54) RADIOLOGY OPERATIONS COMMAND CENTER (ROCC) LOCAL TECHNOLOGIST - SUPERTECHNOLOGIST MATCHING

(71) Applicant: KONINKLIJKE PHILIPS N.V., EINDHOVEN (NL)

(72) Inventors: Hareesh CHAMARTHI, CAMBRIDGE, MA (US); Olga STAROBINETS, NEWTON, MA (US); Sandeep Madhukar DALAL, WINCHESTER, MA (US); Ranjith Naveen TELLIS, TEWKSBURY, MA (US); Yuechen QIAN, LEXINGTON, MA (US)

(21) Appl. No.: 17/924,400

(22) PCT Filed: May 26, 2021
(86) PCT No.: PCT/EP2021/063948

§ 371 Date: Nov. 10, 2022

Related U.S. Application Data
(60) Provisional application No. 62/704,911, filed on Jun. 3, 2020.

(51) Int. Cl. G16H 80/00; G16H 40/20

(52) U.S. Cl. CPC G16H 80/00; G16H 40/20

(57) ABSTRACT

A method (100) of connecting a local medical imaging device operator (LO) with a remote medical imaging expert (RE) during an imaging examination performed using a medical imaging device (2) includes: determining characteristics for available remote medical imaging experts who are available to assist the local operator; matching one or more of the available remote medical imaging experts based on the determined characteristics for the remote medical imaging experts with characteristics of the imaging examination; and providing a user interface (UI) (28, 28′) to at least one display device (24, 24′) operable by the local operator and the remote medical imaging expert, the UI displaying a list (44) of the matched available remote medical imaging experts and via which the local medical imaging device operator can select a matched available medical imaging expert from the displayed list.

The following relates generally to the imaging arts, remote imaging assistance arts, remote imaging examination monitoring arts, and related arts.

### BACKGROUND

Currently, diagnostic imaging is in high demand. As the world population ages, the demand for quick, safe, high quality imaging may only continue to grow, putting further pressure on imaging centers and their staff. In order to perform imaging examinations on patients quickly and safely, maintaining high throughput and quality standards, an imaging provider has to establish an efficient workflow, safeguarding it from disruptions.

Some common workflow disruptions include lack of proper guidance for newly appointed technicians, technicians' lack of experience on imaging machines from various vendors with different control interfaces and features, lack of knowledge of imaging protocols, and so forth. These are examples of some common events that, if encountered at the time of the scan, may adversely impact the current imaging examination, and delay the following exams, potentially disrupting the entire workday. With proper preparation, by providing over the shoulder guidance to the technicians, many of these disruptions could be mitigated or avoided entirely.

The following discloses certain improvements to overcome these problems and others.

### SUMMARY

In one aspect, a non-transitory computer readable medium stores instructions executable by at least one electronic processor to perform a method of connecting a local medical imaging device operator with a remote medical imaging expert during an imaging examination performed using a medical imaging device. The method includes: determining characteristics for available remote medical imaging experts who are available to assist the local operator; matching one or more of the available remote medical imaging experts based on the determined characteristics for the remote medical imaging experts with characteristics of the imaging examination; and providing a user interface (UI) to at least one display device operable by the local operator and the remote medical imaging expert, the UI displaying a list of the matched available remote medical imaging experts and via which the local medical imaging device operator can select a matched available medical imaging expert from the displayed list.

In another aspect, an apparatus for connecting a local medical imaging device operator during an imaging examination performed using a medical imaging device includes: a screen-sharing device for sharing a screen of a controller of the medical imaging device. A telephonic or video communication link is operatively connected with an electronic network for providing telephonic or video communication with a remote medical imaging expert of a set of remote medical imaging experts. A database stores determining characteristics for the remote medical imaging experts of the set of remote medical imaging experts in which the characteristics including at least one of an experience level with a modality of the imaging examination; an experience level with an anatomy of a patient to be imaged during the imaging examination; an experience level of working with the local operator; and an experience level with a type of problem occurring in the imaging examination. At least one electronic processor is programmed to: retrieve, from the database, characteristics for one or more remote medical imaging experts of the set of remote medical imaging experts who are available to assist the local operator in the imaging examination; rank the available remote medical imaging experts based on matching the characteristics of the available remote medical imaging experts with characteristics of the imaging examination; and provide a UI displaying a list of the ranked available remote medical imaging experts, enabling the local operator to select one of the listed available remote medical imaging experts, and establishing an assistance session between the local operator and the selected remote medical imaging expert via the screen-sharing device and the telephonic or video communication link.

In another aspect, a method of connecting a remote medical imaging expert to a local operator to provide assistance during an imaging examination includes: retrieving, from a database, characteristics for one or more remote medical imaging experts available to assist the local operator in an imaging examination; ranking the available remote medical imaging experts with a score indicative of the characteristics of the available remote medical imaging experts with the characteristics of the imaging examination; providing a UI displaying a list of the ranked available remote medical imaging experts; receiving a user input, via at least one user input device, indicative of a selection of one of the listed remote medical imaging experts; collecting data related to an effectiveness of assistance provided by the selected remote medical imaging expert and the local operator; determining one or more quality metrics related to the collected data; and updating the information stored in the database for the selected remote medical imaging expert with the calculated one or more quality metrics.

One advantage resides in providing a remote expert or radiologist assisting a technician in conducting a medical imaging examination with situational awareness of local imaging examination(s) which facilitates providing effective assistance to one or more local operators at different facilities.

Another advantage resides in providing a remote expert or radiologist to assist a technician in conducting a medical imaging examination, in which the remote expert or radiologist is well matched to the imaging modality and imaging examination.

Another advantage resides in preselecting a remote expert or radiologist to assist a technician in conducting an ongoing medical imaging examination, in which the remote expert or radiologist is well matched to the imaging modality and imaging examination, and in which the preselected remote expert or radiologist is prepared to quickly respond to any automatically detected error condition in the ongoing imaging examination.

Another advantage resides in providing a remote expert with information about already-performed steps in a workflow in order to provide assistance for subsequent steps in the workflow.

Another advantage resides in matching a most-suited remote expert for providing assistance to one or more local operators based on an experience level of the remote expert and features of the imaging examination performed by the one or more local operators.

Another advantage resides in providing a remote operator or radiologist with status information on a medical imaging examination using a standard display format that is independent of the controller display of the medical imaging device performing the medical imaging examination.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative apparatus for providing remote assistance in accordance with the present disclosure.

FIG. 2 diagrammatically shows modules implemented by the apparatus of FIG. 1.

FIG. 3 shows an example of an output generated by the apparatus of FIG. 1.

FIG. 4 shows an example flow chart of operations suitably performed by the apparatus of FIG. 1.

### DETAILED DESCRIPTION

The following related to Radiology Operations Command Center (ROCC) systems and methods that provide remote expert or “supertech” assistance to a local technician performing an imaging examination. There is value in providing rapid identification of a well-qualified supertech for assisting in a given imaging examination. Delays in providing the supertech can adversely impact imaging laboratory workflow. Moreover, in some disclosed embodiments, it is contemplated to provide supertech assistance in response to certain automatically detected errors or issues in the imaging examination, which again calls for the supertech to be immediately available upon detection of such an error.

In some embodiments disclosed herein, a supertech matching system matches the best available supertech with the local technician and/or with the current imaging examination. To this end, the system tracks available supertechs. A database stores information on each supertech relating to his/her expertise in various imaging modalities, imaged anatomies, and so forth. For matching to a specific local technician, the database stores similar information for the local technicians. For matching to a specific imaging procedure, information on the imaging procedure is stored. The database may have an ancillary information mining system for obtaining the information on the current imaging examination directly from the imaging scanner controller, or from the Radiology Information System (RIS) or other examination scheduling system.

As the database may store a wide range of features for characterizing the supertechs, local techs, and/or imaging examinations, a feature selection or reduction process may optionally be run for a given matching situation. For example, this may be implemented as a Principal Component Analysis (PCA) to generate highly discriminative features.

Based on the (optionally reduced) set of features, the available supertechs are matched to the local technician and/or to the imaging examination. In one matching approach, a clustering algorithm or other machine learning (ML) component groups the available supertechs by experience in various modalities (e.g., into tech levels 1-5 for a given modality and anatomy) and ranks the available supertechs based on these groupings. In another approach, a (non-machine learning) scoring system is employed to score how well each available supertech matches the local technician and/or examination, and the supertechs are ranked by score.

A user interface (UI) is provided via which the highest-matching supertech is presented to the local tech. In one approach, a list of the top-N highest matching supertechs are provided in a selection dialog, the local tech selects a supertech from the top-N selection dialog of the top N closest matching supertechs, and more detailed information about the selected supertech is displayed in a presentation window of the UI. If the local technician is satisfied with the selected supertech then a “connect” button or the like is selected to initiate an ROCC session with the selected supertech. In a variant of this approach, the UI may be presented to a third-party such as an ROCC administrator who selects the supertech and initiates the ROCC session.

In another variant embodiment, the UI is instead presented to the supertech. This approach may be appropriate in the case of an ROCC session triggered by an automatically detected error condition in an ongoing imaging examination. In this case, the UI would pop up to the highest-ranked supertech, with the displayed information being for the imaging examination in which the error occurred (and possibly also information on the local technician conducting that examination). This serves as an invitation which the supertech can then accept by activating a “connect” button or the like to initiate the ROCC session. Alternatively, the supertech can decline the invitation using a suitable UI dialog selector, in which case the system presents the invitation to the next-highest qualified supertech.

Optionally, the system can collect quality metrics, such as collecting data on whether a given matched supertech was able to effectively assist in the imaging examination. This collected data can be used by a human maintainer who may fine-tune the database features, machine learning component(s) or so forth to optimize performance of the system. In a variant approach, the system can employ adaptive learning such as pattern matching to adaptively adjust the machine learning component(s) to maximize metrics such as the effectiveness of the assistance provided by matched supertechs.

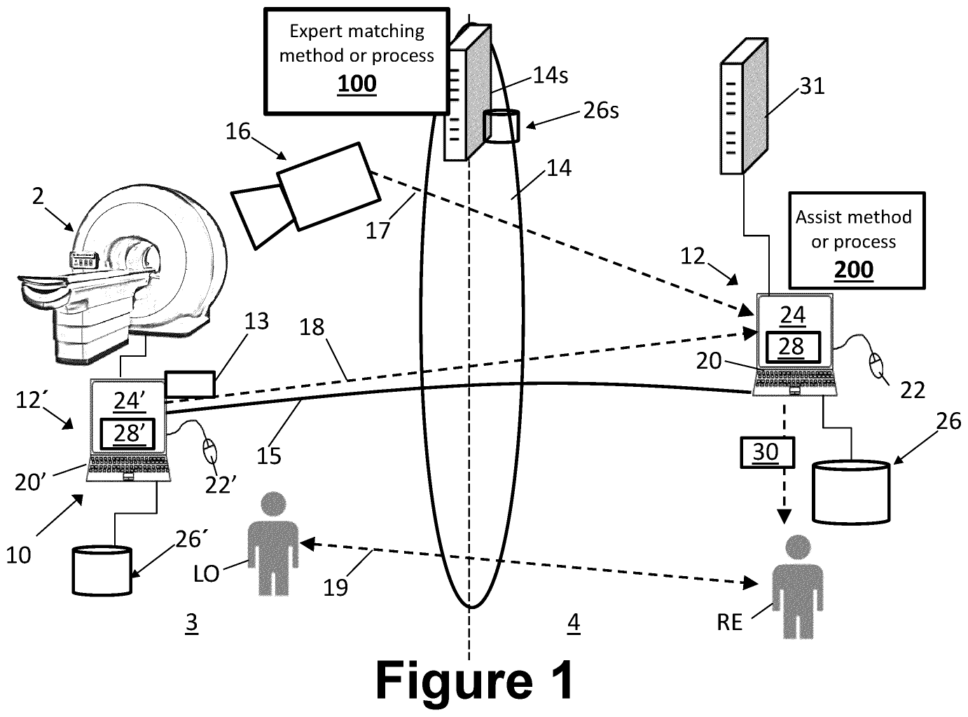

With reference to FIG. 1, an apparatus for providing assistance from a remote medical imaging expert RE (or supertech) to a local technician operator LO is shown. As shown in FIG. 1, the local operator LO, who operates a medical imaging device (also referred to as an image acquisition device, imaging device, and so forth) 2, is located in a medical imaging device bay 3, and the remote operator RE is disposed in a remote service location or center 4. It should be noted that the “remote operator” RE may not necessarily directly operate the medical imaging device 2, but rather provides assistance to the local operator LO in the form of advice, guidance, instructions, or the like. The remote location 4 can be a remote service center, a radiologist's office, a radiology department, and so forth. The remote location 4 may be in the same building as the medical imaging device bay 3 (this may, for example, in the case of a “remote operator” RE who is a radiologist tasked with peri-examination image review), but more typically the remote service center 4 and the medical imaging device bay 3 are in different buildings, and indeed may be located in different cities, different countries, and/or different continents. In general, the remote location 4 is remote from the imaging device bay 3 in the sense that the remote operator RE cannot directly visually observe the imaging device 2 in the imaging device bay 3 (hence optionally providing a video feed as described further herein).

The image acquisition device 2 can be a Magnetic Resonance (MR) image acquisition device, a Computed Tomography (CT) image acquisition device; a positron emission tomography (PET) image acquisition device; a single photon emission computed tomography (SPECT) image acquisition device; an X-ray image acquisition device; an ultrasound (US) image acquisition device; or a medical imaging device of another modality. The imaging device 2 may also be a hybrid imaging device such as a PET/CT or SPECT/CT imaging system. While a single image acquisition device 2 is shown by way of illustration in FIG. 1, more typically a medical imaging laboratory will have multiple image acquisition devices, which may be of the same and/or different imaging modalities. For example, if a hospital performs many CT imaging examinations and relatively fewer MRI examinations and still fewer PET examinations, then the hospital's imaging laboratory (sometimes called the “radiology lab” or some other similar nomenclature) may have three CT scanners, two MRI scanners, and only a single PET scanner. This is merely an example. Moreover, the remote service center 4 may provide service to multiple hospitals. The local operator controls the medical imaging device 2 via an imaging device controller 10. The remote operator is stationed at a remote workstation 12 (or, more generally, an electronic controller 12).

As used herein, the term “medical imaging device bay” (and variants thereof) refer to a room containing the medical imaging device 2 and also any adjacent control room containing the medical imaging device controller 10 for controlling the medical imaging device. For example, in reference to an MRI device, the medical imaging device bay 3 can include the radiofrequency (RF) shielded room containing the MRI device 2, as well as an adjacent control room housing the medical imaging device controller 10, as understood in the art of MRI devices and procedures. On the other hand, for other imaging modalities such as CT, the imaging device controller 10 may be located in the same room as the imaging device 2, so that there is no adjacent control room and the medical bay 3 is only the room containing the medical imaging device 2. In addition, while FIG. 1 shows a single medical imaging device bay 3, it will be appreciated that the remote service center 4 (and more particularly the remote workstation 12) is in communication with multiple medical bays via a communication link 14, which typically comprises the Internet augmented by local area networks at the remote operator RE and local operator LO ends for electronic data communications.

As diagrammatically shown in FIG. 1, in some embodiments, a camera 16 (e.g., a video camera) is arranged to acquire a video stream 17 of a portion of the medical imaging device bay 3 that includes at least the area of the imaging device 2 where the local operator LO interacts with the patient, and optionally may further include the imaging device controller 10. The video stream 17 is sent to the remote workstation 12 via the communication link 14, e.g. as a streaming video feed received via a secure Internet link.

In other embodiments, the live video feed 17 is, in the illustrative embodiment, provided by a video cable splitter 15 (e.g., a DVI splitter, a HDMI splitter, and so forth). In other embodiments, the live video feed 17 may be provided by a video cable connecting an auxiliary video output (e.g. aux vid out) port of the imaging device controller 10 to the remote workstation 12 of the operated by the remote expert RE.

Additionally or alternatively, a screen mirroring data stream 18 is generated by a screen sharing device 13, and is sent from the imaging device controller 10 to the remote workstation 12. The communication link 14 also provides a natural language communication pathway 19 for verbal and/or textual communication between the local operator and the remote operator. For example, the natural language communication link 19 may be a Voice-Over-Internet-Protocol (VOIP) telephonic connection, an online video chat link, a computerized instant messaging service, or so forth. Alternatively, the natural language communication pathway 19 may be provided by a dedicated communication link that is separate from the communication link 14 providing the data communications 17, 18, e.g. the natural language communication pathway 19 may be provided via a landline telephone.

FIG. 1 also shows, in the remote service center 4 including the remote workstation 12, such as an electronic processing device, a workstation computer, or more generally a computer, which is operatively connected to receive and present the video 17 of the medical imaging device bay 3 from the camera 16 and to present the screen mirroring data stream 18 as a mirrored screen. Additionally or alternatively, the remote workstation 12 can be embodied as a server computer or a plurality of server computers, e.g. interconnected to form a server cluster, cloud computing resource, or so forth. The workstation 12 includes typical components, such as an electronic processor 20 (e.g., a microprocessor), at least one user input device (e.g., a mouse, a keyboard, a trackball, and/or the like) 22, and at least one display device 24 (e.g. an LCD display, plasma display, cathode ray tube display, and/or so forth). In some embodiments, the display device 24 can be a separate component from the workstation 12. The display device 24 may also comprise two or more display devices, e.g. one display presenting the video 17 and the other display presenting the shared screen of the imaging device controller 10 generated from the screen mirroring data stream 18. Alternatively, the video and the shared screen may be presented on a single display in respective windows. The electronic processor 20 is operatively connected with a one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 12, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a graphical user interface (GUI) 28 for display on the remote operator display device 24.

The medical imaging device controller 10 in the medical imaging device bay 3 also includes similar components as the remote workstation 12 disposed in the remote service center 4. Except as otherwise indicated herein, features of the medical imaging device controller 10, which includes a local workstation 12″, disposed in the medical imaging device bay 3 similar to those of the remote workstation 12 disposed in the remote service center 4 have a common reference number followed by a “prime” symbol, and the description of the components of the medical imaging device controller 10 will not be repeated. In particular, the medical imaging device controller 10 is configured to display a GUI 28′ on a display device or controller display 24″ that presents information pertaining to the control of the medical imaging device 2, such as configuration displays for adjusting configuration settings an alert 30 perceptible at the remote location when the status information on the medical imaging examination satisfies an alert criterion of the imaging device 2, imaging acquisition monitoring information, presentation of acquired medical images, and so forth. It will be appreciated that the screen mirroring data stream 18 carries the content presented on the display device 24′ of the medical imaging device controller 10. The communication link 14 allows for screen sharing between the display device 24 in the remote service center 4 and the display device 24″ in the medical imaging device bay 3. The GUI 28″ includes one or more dialog screens, including, for example, an examination/scan selection dialog screen, a scan settings dialog screen, an acquisition monitoring dialog screen, among others. The GUI 28″ can be included in the video feed 17 or the mirroring data stream 17″ and displayed on the remote workstation display 24 at the remote location 4.

FIG. 1 also shows the remote workstation 12 in communication with a database 31 storing patient information (e.g., an electronic health record (EHR) database, an electronic medical record (EMR) database, a Radiology Information System (RIS) database, and so forth).

FIG. 1 shows an illustrative local operator LO, and an illustrative remote expert RE (i.e. expert, e.g. supertech). However, in a Radiology Operations Command Center (ROCC) as contemplated herein, the ROCC provides a staff of supertechs who are available to assist a local operator LO at different hospitals, radiology labs, or the like. The ROCC may be housed in a single physical location, or may be geographically distributed. For example, in one contemplated implementation, the remote operators RO are recruited from across the United States and/or internationally in order to provide a staff of supertechs with a wide range of expertise in various imaging modalities and in various imaging procedures targeting various imaged anatomies. In view of this multiplicity of local operators LO and multiplicity of remote operators RO, the disclosed communication link 14 includes a server computer 14s (or a cluster of servers, cloud computing resource comprising servers, or so forth) which is programmed to establish connections between selected local operator LO/remote expert RE pairs. For example, if the server computer 14s is Internet-based, then connecting a specific selected local operator LO/remote expert RE pair can be done using Internet Protocol (IP) addresses of the various components 16, 10, 12, the telephonic or video terminals of the natural language communication pathway 19, et cetera. The server computer 14s is operatively connected with a one or more non-transitory storage media 26s. The non-transitory storage media 26s may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the server computer 14s, various combinations thereof, or so forth. It is to be understood that any reference to a non-transitory medium or media 26s herein is to be broadly construed as encompassing a single medium or multiple media of the same or different types. Likewise, the server computer 14s may be embodied as a single electronic processor or as two or more electronic processors. The non-transitory storage media 26s stores instructions executable by the server computer 14s. In addition, the non-transitory computer readable medium 26s (or another database) stores data related to a set of remote experts RE and/or a set of local operators LO. The remote expert data can include, for example, skill set data, work experience data, data related to ability to work on multi-vendor modalities, data related to experience with the local operator LO and so forth.

Furthermore, as disclosed herein the server 14s performs an expert matching method or process 100 that matches an available well-qualified remote expert RE with a given local operator LO.

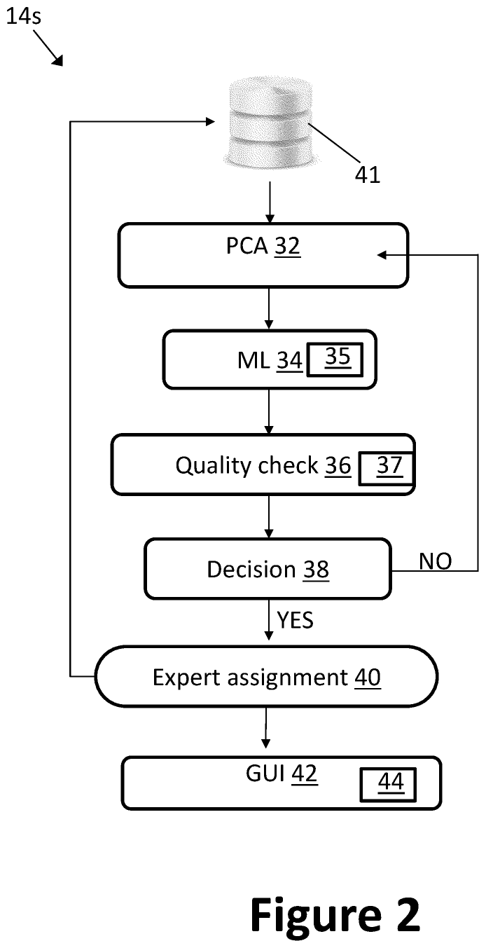

Referring now to FIG. 2, and with continuing reference to FIG. 1, in one embodiment of the expert matching method or process 100, the server 14s is programmed with several components to provide assistance to the remote expert RE. A principal component analysis model 32 is configured to analyze the remote expert data stored in a database 41 (which could be the non-transitory computer readable medium 26s) and identify features to perform a medical imaging examination, such as a modality of the medical imaging device 2, a vendor of the medical imaging device, a type of protocol to be used in the medical imaging examination, anatomy to be imaged, condition of the patient to be imaged, prior work experience of the local operator LO in handling relevant cases, prior work experience of the remote expert RE in handling relevant cases an availability of the remote experts, and so forth. In particular, the availability of each remote expert RE is retrieved. In some examples, the principal component analysis model 32 can be a machine-learning (ML) model.

The principal component analysis model 32 is configured to categorize the remote experts RE into skill levels on a scale of level 1-5. A “level 1 expert” can have around 0-2 years of experience be well-skilled in patient care and safety, and will need support and guidance in perfecting basics in obtaining images (e.g., from a more experienced super-tech). A “level 2 expert” can have around 2-3 years of experience, be well-skilled in patient care and safety, able to produce quality images, and will need to solidify knowledge to get confidence making independent decisions and exposure to new, more challenging cases. A “level 3 expert” has at least 3-5 years of experience, and has a strong ability in obtaining standard images. A “level 4 expert” has 5 or more years of experience, has a strong knowledge on advance imaging, can advise on examination cards, can anticipate most imaging protocols, and should be able to maintain and up-to-date relationship with other technicians on preferences. A “level 5 expert” has at least 10 years of experience, has strong knowledge on advance medical imaging, can set exam cards, can anticipate all imaging protocols, should be able to support on retaining all the knowledge to help local operators LO, and should be able to maintain up-to-date relationship with technologists on preferences.

Data characterizing the remote experts RE can be provided, as input to the principal component analysis model 32. To do so, the remote experts RE can for example fill out questionnaires when joining the ROCC to provide information on their experience with different modalities, imaged anatomies, etc. In one embodiment, the principal component analysis model 32 includes, for example, five features for use in the model, where the features are calculated based on based on relevant work experience of the remote experts, and corresponding medical imaging examination complexity conditions. For example, one set of features could be obtained from a work experience of super-tech (labelled as ST-WE) combined with a complexity of the medical imaging examination to produce a binarized feature. If the first quartile, median, and third quartiles of the ST-WE and relativity to exam complexity (REC) or support required to perform the scan, are 10, 20, and 30, the following four features can be defined: “Is the ST-WE_REC value less than 10?” “Is the ST-WE_REC value greater than or equal to 10?”; “Is the ST-WE_REC value greater than or equal to 20?” and “Is the ST-WE_REC value greater than or equal to 30?”. Other binarized features can be similarly obtained, such as ST-WE_ScannerType, ST-WE_Vendor, and so on. Other constraints to the principal component analysis model 32 can include constraints on accuracies such as an area under the curve (AUC) value.

A ML module 34 is configured to group all of the remote experts RE of the set of remote experts into various categories based on, for example their experience in working with different imaging modalities, patient condition, medical imaging examination complexity, modality, modality vendor, local operator LO experience, etc. One or more ML models 35 can be generated and used to predict a set of variables relevant for the medical imaging examination. The models 35, along with the principal component analysis model 32, and the remote expert RE data stored in the non-transitory computer readable medium 26 are used to find an “best-fit” remote expert to assist the local operator LO in the medical imaging examination. For example, the models 35 can be used to prioritize the remote experts RE into the following categories: exact match, relevant experience, exact examination experience but no modality experience, modality experience but no examination experience, and no modality or examination experience.

A quality metric check module 36 is configured simulate remote expert RE—local operator LO pairings make future pairing predictions, and also monitor quality metric values 37 such as the AUC values from the principal component analysis model 32 and the models 35.

A decision-making module 38 is configured to determine whether results from the ML module 34 and the quality metric check module 36 meet a predetermined satisfactory threshold. If the threshold is not met, then the remote expert RE can change the input constraints to the principal component analysis model 32 and re-obtain a new set of remote experts based on the new input constraints, along with a new set of quality metric values 37.

If the threshold is met, then an assignment module 40 is configured to identify the best remote expert RE (or a ranked top-N list of N most highly ranked experts RE) for the medical imaging examination (e.g., selecting accurate sequences for imaging and obtain high-quality images successfully) and match the best remote expert with the local operator LO. If a match is made, then the generated models 35 and quality metric values 37 can be stored in the database 41 (or, alternatively, in the non-transitory computer readable medium 26) for use in future matchings.

A GUI output module 42 is configured to output a list 44 on the display device 24 of the remote workstation 12 (via the GUI 28) and/or the display device 24″ of the medical imaging device controller 10 (via the GUI 28″) with the best available remote experts RE, along with one or more corresponding quality metric values 37 (e.g., confidence, values, AUC values, and so forth). The list 44 can include only a set number of best available remote experts RE, such as the three best available experts and the corresponding quality metric values 37.

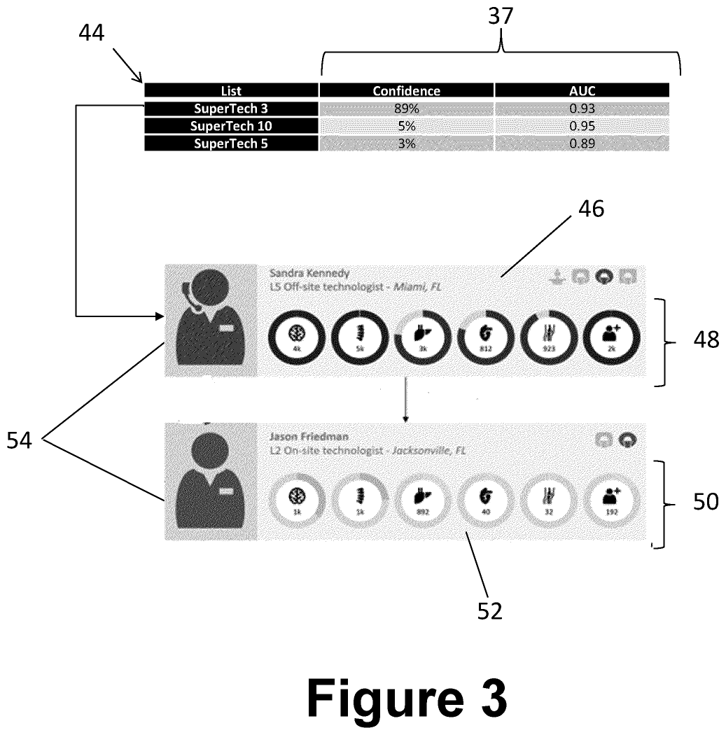

FIG. 3 shows an example of a list 44. As shown in FIG. 3, the list 44 includes three best available remote experts RE, along with the corresponding quality metric values 37 including confidence values and AUC values. Upon a selection of one of the listed remote experts RE via the at least one user input device 22, 22″, a drop-down menu 46 listing a set of experience metrics 48 of the remote expert RE can be shown. As shown in FIG. 3, the set of experience metrics 48 can include a brain imaging experience metric, a spine imaging experience metric, a liver imaging experience metric, a heart imaging experience metric, a knee imaging experience metric, and a whole-body imaging experience metric. These metrics 48 are compared to corresponding experience metrics 50 shown on a drop-down menu 52 of a local operator LO. From this, the best available remote expert RE can be selected to assist the local operator LO. In addition, the list 44 can also include a communication button 54 selectable by the remote expert RE or the local operator LO to establish the natural language communication pathway 19 via the communication link 14 between the two parties.

Upon selection of a remote expert RE to provide assistance to a given local operator LO, the communication link 14 connects the local operator LO/selected remote expert RE. The remote workstation 12 of the selected remote expert RE, and/or the medical imaging device controller 10 being run by the local operator LO, is configured to perform a method or process 200 for providing assistance from the remote expert RE to the local operator LO. For brevity, the method 200 will be described as being performed by the remote workstation 12. The non-transitory storage medium 26 stores instructions which are readable and executable by the at least one electronic processor 20 (of the workstation 12, as shown, and/or the electronic processor or processors of a server or servers on a local area network or the Internet) to perform disclosed operations including performing the method or process 200.

A suitable implementation of the assistance method or process 200 is as follows. The method 200 is performed over the course of (at least a portion of) a medical imaging examination performed using the medical imaging device 2, and the local expert RE is one selected via the matching method 100. As used herein, the term “duration of a medical imaging examination” (or variants thereof) refers to a time period of a medical imaging examination that includes (i) an actual image acquisition time, (ii) imaging follow-on processing time, and (iii) up to a time of patient release. To perform the method 200, the workstation 12 in the remote location 4 is programmed to receive at least one of: (i) the video 17 from the video camera 16 of the medical imaging device 2 located in the medical imaging device bay 3; and/or (ii) the screen sharing 18 from the screen sharing device 19; and/or (iii) the video 17 tapped by the video cable splitter 15. The video feed 17 and/or the screen sharing 18 can be displayed at the remote workstation display 24, typically in separate windows of the GUI 28. The video feed 17 and/or the screen sharing 18 can be screen-scraped to determine information related to the medical imaging examination (e.g., modality, vendor, anatomy to be imaged, cause of issue to be resolved, and so forth). In particular, the GUI 28 presented on the display 24 of the remote workstation 12 preferably includes a window presenting the video 17, and a window presenting the mirrored screen of the medical imaging device controller 10 constructed from the screen mirroring data stream 18, and status information on the medical imaging examination that is maintained at least in part using the screen-scraped information. This allows the remote operator RE to be aware of the content of the display of the medical imaging device controller 10 (via the shared screen) and also to be aware of the physical situation, e.g. position of the patient in the medical imaging device 2 (via the video 17), and to additionally be aware of the status of the imaging examination as summarized by the status information. During an imaging procedure, the natural language communication pathway 19 is suitably used to allow the local operator LO and the remote operator RE to discuss the procedure and in particular to allow the remote operator to provide advice to the local operator.

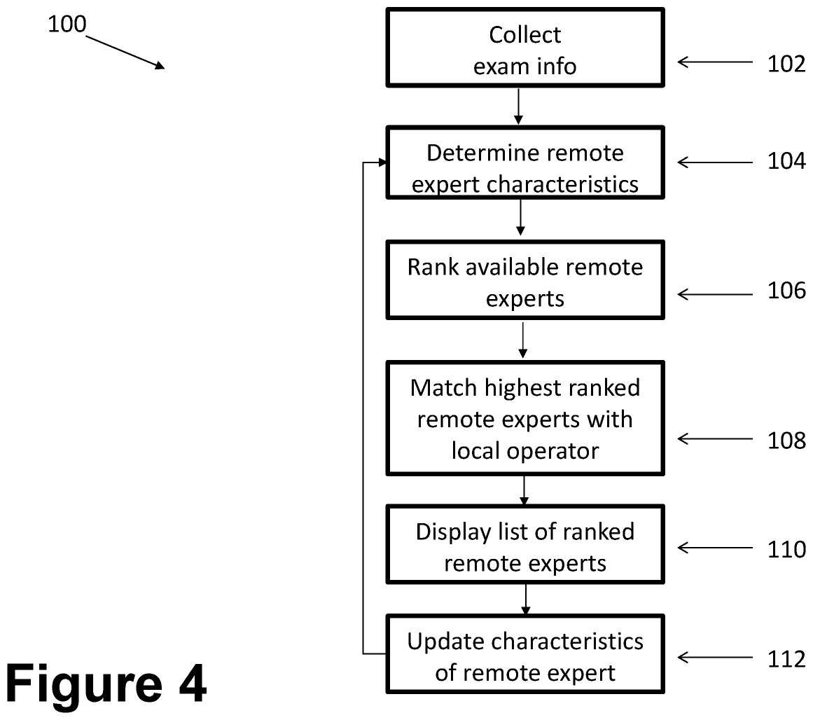

With reference to FIG. 4, and with continuing reference to FIGS. 1-3, an illustrative embodiment of the expert matching method 100 is diagrammatically shown as a flowchart. At an operation 102, information on the medical imaging examination to be performed by the local operator LO is collected. In one approach, the information is communicated to the server 14s by an imaging laboratory scheduling system that scheduled the imaging examination. In another contemplated approach, the local operator LO fills out an electronic expert assistance request form pushed to the local operator LO by the server 14s (e.g. as a webpage), and the form asks for relevant information about the imaging examination (e.g. modality, vendor, anatomy to be imaged, cause of issue to be resolved, and so forth). In yet another contemplated approach, the video feed 17 and/or the screen sharing 18 is captured at the server 14s and is screen-scraped to determine information related to the medical imaging examination (e.g., modality, vendor, anatomy to be imaged, cause of issue to be resolved, and so forth). In some examples, the information collected during the operation 102 can include availability of different remote experts RE.

At an operation 104, characteristics for available remote medical imaging experts RE who are available to assist the local operator LO are determined. To do so, the remote expert data stored in the non-transitory computer readable medium 26 can be retrieved and input to the principal component analysis model 32. A feature selection process, such as a Principal Component Analysis (PCA) can be performed on the retrieved data and the screen-scraped information from the video 17 and the screen sharing 18 to generate the principal component analysis model 32 and determine the characteristics of the available remote experts RE. The characteristics of the available remote experts RE can include at least one of: an experience level with an imaging modality of the imaging examination; an experience level with an anatomy of a patient to be imaged during the imaging examination; an experience level of working with the local operator LO; an experience level with a type of problem occurring in the imaging examination, and so forth. In some examples, the availability data collected at the operation 102 can be combined with scheduling information of the imaging examination, to not only determine which remote experts RE are available, but the duration of the availability. This allows comparison of the expected duration of the imaging examination with the duration of availability of remote experts, so as to avoid a situation in which the remote expert becomes unavailable before the imaging examination (including follow-on processing time, up to patient release) is completed. This reduces likelihood that the patient will not need to re-visit. A list of “soon-to-be available remote experts RE can also be included.

At an operation 106, the available remote experts RE are ranked. To do so, a ML operation is applied to the principal component analysis model 32 to rank the available remote experts RE. This is performed using the ML module 34 and the generated models 35. In addition, the quality metric values 37 are generated using the quality metric check module 38, which are also used to rank the available remote experts RE. The quality metric values 37 are used as scores to rank the remote experts RE. The decision making module 38 then ranks the available remote experts RE based on the quality metrics 37 and the results of the models 35 generated by the ML module 34.

At an operation 108, one or more of the available remote experts RE are matched with the local operator LO performing the medical imaging examination. This is performed using the assignment module 40. The best remote expert RE for the medical imaging examination (e.g., selecting accurate sequences for imaging and obtain high-quality images successfully) and match the best remote expert with the local operator LO.

At an operation 110, the list 44 of ranked available remote experts RE is displayed via the GUI 28 on the display device 24 of the remote workstation 12 and/or via the GUI 28′ on the display device 24′ of the medical imaging device controller 10. Referring back to FIG. 2, the list 44 can include the quality metrics 37 as scores used to rank the available remote experts RE. The quality metrics 37 can be displayed with the corresponding remote expert RE, and the remote experts can be ranked according to the highest quality metrics. The list 44 can also include “soon-to-be available remote experts RE based on the schedule availability data.

At an operation 110, one of the remote experts RE listed on the list 44 is selected by the local operator LO. To do so, the local operator LO uses the at least one user input device 22′ and selects one of the listed remote experts RE. In some examples, the local operator LO can select the remote expert RE to open the drop down menu 46 to show the selected remote expert's set of experience metrics 48. The local operator LO can also select the communication button 54 selectable by the remote expert RE or the local operator LO to establish the natural language communication pathway 19 via the communication link 14 between the two parties so that the screens of the medical imaging device controller 10 and the remote workstation 12 are shared. In some examples, the selected remote expert RE can decline the natural language communication pathway 19 (e.g., if the remote expert RE is busy, or feels that the problem to be addressed in the medical imaging examination does not suit his or her skills). In this case, the communication link can be established between another one of the listed remote experts RE (e.g., the next highest ranked remote expert). This process can continue until one of the remote experts RE accepts the natural language communication pathway 19.

At an optional operation 112, the database 41 can be updated based on the interactions between the selected remote expert RE and the local operator LO. In one example, if the selected remote expert RE declines to help the local operator LO, the database 41 can be updated to no longer recommend that remote expert for those types of problems, for that particular local operator LO, and so forth. In addition, data can be collected related to an effectiveness of assistance provided by the selected remote expert RE and the local operator LO. This can be done via a feedback form filled out by the local operator LO and/or the remote expert RE. In one example, one or more of the quality metrics 37 for the selected remote expert RE can be re-calculated, and stored in the database 41 (along with updates related to the additions to the remote expert's experience through handling the matter with the local operator LO). In another example, an adaptive-learning process on the collected data to maximize the quality metrics 37 related to the effectiveness of the assistance, which can also be stored in the database 41.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions executable by at least one electronic processor to perform a method of connecting a local medical imaging device operator (LO) with a remote medical imaging expert (RE) during an imaging examination performed using a medical imaging device, the method comprising:
determining characteristics for available remote medical imaging experts who are available to assist the local operator;
matching one or more of the available remote medical imaging experts based on the determined characteristics for the remote medical imaging experts with characteristics of the imaging examination; and
providing a user interface (UI) to at least one display device operable by the local operator and the remote medical imaging expert, the UI displaying a list of the matched available remote medical imaging experts and via which the local medical imaging device operator can select a matched available medical imaging expert from the displayed list.

2. The non-transitory computer readable medium of claim 1, wherein the method further includes:
ranking the available remote medical imaging experts (RE) based on a score of the characteristics of the available remote medical imaging experts and the characteristics of the imaging examination.

3. The non-transitory computer readable medium of claim 2, wherein providing the UI displaying the list of the matched available remote medical imaging experts (RE) includes:
providing the list as a ranked list of the available remote medical imaging experts according the scores.

4. The non-transitory computer readable medium of claim 2, wherein providing the UI displaying the list of the matched available remote medical imaging experts (RE) includes:
providing the list including the highest ranked available remote medical imaging experts according the scores.

5. The non-transitory computer readable medium of claim 3, wherein providing the UI displaying the list of the matched available remote medical imaging experts (RE) includes:
listing the scores of the listed available remote medical imaging experts on the UI.

6. The non-transitory computer readable medium of claim 3, wherein the determining of the characteristics for available remote medical imaging experts (RE) includes determining a scheduling availability of the remote medical imaging experts; and the providing of the UI displaying the list includes providing the list with the scheduling availability of the remote medical imaging experts.

7. The non-transitory computer readable medium of claim 2, wherein ranking the available remote medical imaging experts (RE) includes:
applying a machine learning (ML) operation to the characteristics of the available remote medical imaging experts to rank the available remote medical imaging experts.

8. The non-transitory computer readable medium of claim 1, wherein determining characteristics for remote medical imaging experts (RE) of a set of remote medical imaging experts available to assist the local operator (LO) includes:
performing a feature selection process on the characteristics of the remote medical imaging experts and the characteristics of the imaging examination.

9. The non-transitory computer readable medium of claim 8, wherein the feature selection process is a Principal Component Analysis (PCA) process.

10. The non-transitory computer readable medium of claim 1, wherein the characteristics of the available remote medical imaging experts (RE) include at least one of:
an experience level with an imaging modality of the imaging examination;
an experience level with an anatomy of a patient to be imaged during the imaging examination;
an experience level of working with the local operator (LO); and
an experience level with a type of problem occurring in the imaging examination.

11. The non-transitory computer readable medium of claim 1, wherein providing the UI displaying a list of the matched available remote medical imaging experts (RE) includes:
upon receiving a user input via at least one user input device indicative of a selection of one of the listed remote medical imaging experts, providing a drop-down menu displaying information about the selected remote medical imaging expert.

12. The non-transitory computer readable medium of claim 1, wherein providing the UI displaying a list of the matched available remote medical imaging experts (RE) includes:
upon receiving a user input via at least one user input device indicative of a selection of one of the listed remote medical imaging experts, establishing a two-way telephonic or video communication link between the selected remote medical imaging expert and the local operator (LO) and sharing a display of the medical imaging device at a workstation of the selected remote medical imaging expert.

13. The non-transitory computer readable medium of claim 12, wherein providing the UI displaying a list of the matched available remote medical imaging experts (RE) includes:
upon receiving an indication from the selected remote medical imaging expert of a decline of the communication link, establishing a communication link between another one of the listed remote medical imaging experts and the local operator (LO).

14. The non-transitory computer readable medium of claim 1, wherein the characteristics of the remote medical imaging experts (RE) are stored in a database, and the method further includes:
collecting data related to an effectiveness of assistance provided by the selected remote medical imaging expert and the local operator (LO);
calculating one or more quality metrics related to the collected data; and
updating the information stored in the database for the selected remote medical imaging expert with the calculated one or more quality metrics.

15. The non-transitory computer readable medium of claim 14, wherein the method further includes:
collecting data related to an effectiveness of assistance provided by the selected remote medical imaging expert (RE) and the local operator (LO);
performing an adaptive-learning process on the collected data to maximize quality metrics related to the effectiveness of the assistance; and
updating the information stored in the database for the selected remote medical imaging expert with the maximized quality metrics.

16. An apparatus for connecting a local medical imaging device operator (LO) during an imaging examination performed using a medical imaging device, the apparatus comprising:
a screen-sharing device for sharing a screen of a controller of the medical imaging device;
a telephonic or video communication link operatively connected with an electronic network for providing telephonic or video communication with a remote medical imaging expert (RE) of a set of remote medical imaging experts;
a database storing determining characteristics for the remote medical imaging experts of the set of remote medical imaging experts, the characteristics including at least one of: an experience level with a modality of the imaging examination; an experience level with an anatomy of a patient to be imaged during the imaging examination; an experience level of working with the local operator; and an experience level with a type of problem occurring in the imaging examination; and
at least one electronic processor programmed to:
retrieve, from the database, characteristics for one or more remote medical imaging experts of the set of remote medical imaging experts who are available to assist the local operator in the imaging examination;
rank the available remote medical imaging experts based on matching the characteristics of the available remote medical imaging experts with characteristics of the imaging examination; and
provide a user interface (UI) displaying a list of the ranked available remote medical imaging experts, enabling the local operator to select one of the listed available remote medical imaging experts, and establishing an assistance session between the local operator and the selected remote medical imaging expert via the screen-sharing device and the telephonic or video communication link.

17. The apparatus of claim 16, wherein the at least one electronic processor is further programmed to:
upon receiving a user input via at least one user input device indicative of a selection of one of the listed remote medical imaging experts (RE), establishing a communication link between the selected remote medical imaging expert and the local operator (LO).

18. The apparatus of claim 16, wherein the at least one electronic processor is further programmed to:
apply a machine learning (ML) operation to the characteristics of the available remote medical imaging experts (RE) to rank the available remote medical imaging experts.

19. The apparatus of claim 16, wherein the at least one electronic processor is further programmed to:
upon receiving a user input via at least one user input device indicative of a selection of one of the listed remote medical imaging experts, provide a drop-down menu displaying information about the selected remote medical imaging expert.

20. A method of connecting a remote medical imaging expert (RE) to a local operator (LO) to provide assistance during an imaging examination, the method including:
retrieving, from a database, characteristics for one or more remote medical imaging experts available to assist the local operator in an imaging examination;
ranking the available remote medical imaging experts with a score indicative of the characteristics of the available remote medical imaging experts with the characteristics of the imaging examination;
providing a user interface (UI) displaying a list of the ranked available remote medical imaging experts;
receiving a user input, via at least one user input device, indicative of a selection of one of the listed remote medical imaging experts;
collecting data related to an effectiveness of assistance provided by the selected remote medical imaging expert and the local operator;
determining one or more quality metrics related to the collected data; and
updating the information stored in the database for the selected remote medical imaging expert with the calculated one or more quality metrics.
